(19) United States
(12) Patent Application Publication
(10) Pub. No.: US 2025/0299333 A1
(43) Pub. Date: Sep. 25, 2025

(54) Methods and Products for Bladder Cancer Grading

(71) Applicants: Queen's University at Kingston, Kingston (CA); Kingston Health Sciences Centre, Kingston (CA)

(72) Inventors: Katherine Lindale, Ottawa (CA); Ava Slotman, New Westminster (CA); David M. Berman, Kingston (CA); Robert Gooding, Kingston (CA); Amber Simpson, Kingston (CA); Kathrin Tyryshkin, Kingston (CA)

(21) Appl. No.: 19/089,714

(22) Filed: Mar. 25, 2025

Related U.S. Application Data
(60) Provisional application No. 63/569,467, filed on Mar. 25, 2024.

(51) Int. Cl. G06T 7/00; G06T 7/11; G06V 10/26; G06V 10/77; G06V 20/69; G16H 50/30

(52) U.S. Cl. CPC G06T 7/0012; G06T 7/11; G06V 10/26; G06V 10/7715; G06V 20/695; G06V 20/698; G16H 50/30; G06T 2207/10056; G06T 2207/20081; G06T 2207/30024; G06T 2207/30096; G06V 2201/03

(57) ABSTRACT

A computer-implemented method for classifying cancer uses image analysis software to analyze a digital histology image of tumour cells of a patient. The image analysis software is trained to segment tissue regions, identify nuclei, and measure nuclear features of a plurality of nuclei. Summary statistics for nuclear feature values are obtained and one or multiple prognostic classifiers are applied to the patient's nuclear feature values to produce a prognostic score for the patient from the prognostic classifiers. The prognostic score may be for recurrence-free survival of the patient or may discriminate between high-grade and low-grade tumours.

### RELATED APPLICATION

This application claims the benefit of the filing date of Application No. 63/569,467 filed on Mar. 25, 2024, the contents of which are incorporated herein by reference in their entirety.

### FIELD

The invention relates generally to histopathologic grading in cancer to determine prognosis. More specifically, the invention relates to development and use of grade-based quantified nuclear features in prognostic models of cancer.

### BACKGROUND

Bladder cancer (BC) is the tenth most common cancer worldwide, with 70% of bladder cancer patients diagnosed with non-muscle invasive bladder cancer (NMIBC). BC has the highest lifetime cost of all cancers with longitudinal treatment and monitoring regimens that are burdensome for the healthcare system and detrimental to patient quality of life. Histologic grade is a key factor in patient risk stratification and care decision-making based on cellular and structural tumour features that indicate tumour aggression.

The current NMIBC grading system is highly subjective and does not adequately categorize patients by risk of cancer recurrence or progression, frequently leading to overtreatment and excessive monitoring [1]. Further, the pathology workflow lacks a feedback loop, meaning pathologists do not receive patient outcome data, and as such, cannot detect associations between histologic grading features and meaningful endpoints such as recurrence or progression.

There are three T stages of NMIBC: Tis, Ta, and T1. In Tis, also called carcinoma in situ (CIS), there are very early cancer cells in the inner layer of the bladder lining. The cancer cells look very abnormal and are likely to grow quickly. This is referred to as high grade. In Ta the tumour is papillary in configuration and confined to the innermost layer of the bladder lining. In T1 the cancer has started to grow into the connective tissue beneath the bladder lining.

For patients diagnosed with stage Ta NMIBC, stage progression occurs in 4.4% of low-grade (LG) cases and 14.1% of high-grade (HG) cases, while recurrence occurs in 52% of LG cases and 60.5% of HG cases⁶. These statistics highlight the limited prognostic value of the current grading system, as it provides only marginal utility in predicting recurrence and progression. This inadequacy contributes significantly to the uncertainty surrounding an NMIBC diagnosis, often leading to frequent, long-term, and reactive care. However, it also suggests that refinements to the grading system could substantially enhance the ability to predict recurrence and progression risk, leading to more personalized and effective management of the disease.

Recurrence is a major clinical inflection point of interest that impacts patient quality of life. The time it takes for an NMIBC to recur is highly unpredictable, requiring patients to return frequently for surveillance cystoscopies several years after the initial tumour has been resected and treated. The shortcomings in the current grading system present an opportunity to improve future outcomes and reduce the economic and social costs of NMIBC usage by re-defining risk thresholds based on meaningful clinical outcomes.

Prognostic models in NMIBC and across cancers generally seek to either discover novel biomarkers based on potential biological mechanisms, or to validate the utility of existing clinical standard risk scoring strategies.

Surgical pathologists interpret histologic snapshots of dynamic tumour growth to estimate how quickly the cancer is growing and spreading based on how disordered, variably sized and misshapen the nuclei appear. As such, histologic grading features are intended to be a visually interpretable representation of the underlying tumour biology. Computational tools have the capacity to process and analyse high volumes of microscopic cellular imaging data objectively and consistently. Therefore, the challenge of establishing a mathematical definition of natural cellular growth patterns, and quantifying cancer's deviations from these patterns, is being approached collaboratively across the globe using digital pathology.

With the intention of simplifying clinical decision making using defined thresholds and cut-off points, an increasing number of classification models for cancer prognosis have been proposed [2-4]. However, harmful data loss occurs when stratifying outcome data by whether or not patients experience the outcome of interest during the study period, or by whether they experience the outcome before a timepoint of interest, for example recurrence within two years of diagnosis. Further, such binary classification models do not account for right-censoring, when patients do not experience the outcome of interest during their observation period [5].

### SUMMARY

One aspect of the invention relates to a computer-implemented method for classifying cancer, comprising: utilizing image analysis software to analyze a digital histology image of tumour cells of a patient, the image analysis software being trained to segment tissue regions, identify cancer nuclei, and measure nuclear features of a plurality of cancer nuclei; obtaining summary statistics for nuclear feature values; applying one or multiple prognostic classifiers to the patient's nuclear feature values; and producing a prognostic score for the patient from the prognostic classifiers.

The nuclear features may be selected from size, shape, texture, and mitotic index.

In one embodiment the nuclear features for nuclei in an image are reduced into summary statistics including one or more of mean, standard deviation, and interquartile range.

In one embodiment the nuclear features for nuclei in an image are used to establish a percent of abnormality of at least one nuclear feature.

In one embodiment cancer and non-cancer regions are segmented, wherein only cancer cell nuclei are included in prognostic classifiers.

In various embodiments the digital histology image my contain at least 500 identifiable cancer cells.

One embodiment comprises using multiple histology images for a single patient and the summary statistics of the nuclear features are a weighted average of the multiple histology images.

In various embodiments the prognostic score is for recurrence-free survival of the patient.

In one embodiment the cancer is non-muscle invasive bladder cancer.

In one embodiment the one or more prognostic classifier comprises a Cox Proportional Hazards (CPH) model.

In one embodiment the one or more prognostic classifier comprises a Random Survival Forest (RSF) model.

In one embodiment the one or more prognostic classifier comprises an interquartile range (IQR)-based outlier detector.

In one embodiment the IQR-based outlier detector determines an outlier score indicating a percent of abnormal size for at least one nuclear size feature.

The prognostic score for the patient may be used to determine the appropriateness and type of treatment for the patient.

Another aspect of the invention relates to non-transitory computer readable media for use with a processor, the computer readable media having stored thereon instructions that when executed by the processor, cause the processor to execute processing steps comprising executing an image analysis algorithm to analyze a digital histology image of tumour cells of a patient, the image analysis algorithm being trained to segment tissue regions, identify cancer nuclei, and measure nuclear features of a plurality of cancer nuclei; determining summary statistics for nuclear feature values; applying one or more prognostic classifiers to the nuclear feature values; and producing a prognostic score for the patient from the prognostic classifiers.

The non-transitory computer readable media may execute processing steps to carry out functions corresponding to methods as described herein.

The non-transitory computer readable media may determine the prognostic score for the patient wherein the prognostic score is used to determine the appropriateness and type of treatment for the patient.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIGS. 1A and 1B are diagrammatic representations of methodologies according to various embodiments.

FIG. 2 is a marked-up digital photomicrographic image of a non muscle-invasive bladder cancer tissue section showing morphometric features: A, mitotic figure; B, using the pixelated contour line (white), area, perimeter, and form factor (4*η*area/perimeter2); C, lesser and largest-diameters of the nucleus to the perimeter; D, major and minor axes (length and width measurements to the nearest ellipse), ellipticalness, and eccentricity; E, solidity and convexity (area (or perimeter)/bounding polygon size, as determined by polygon simplification), according to one embodiment.

FIG. 3 is a flow diagram of a data preprocessing and exclusion strategy, wherein data include NMIBC tissue images and associated nuclear measurement data and clinical data, according to one embodiment.

FIG. 4 is a Kaplan-Meier curve for 163 stage Ta NMIBC patients grouped by pathologist-assigned grade.

FIG. 5 is a Kaplan-Meier curve for 163 stage Ta NMIBC patients grouped by American Urological Association (AUA) risk score at earliest transurethral resection of a bladder tumour (TURBT).

FIG. 6 is a Kaplan-Meier curve of histologic prognostic score as determined by a CPH model, with the KHSC NMIBC survival cohort (n=163) split by median prognostic score, wherein features included in the model were mitotic index, mean variance hematoxylin (HEM, a measure of staining intensity for nuclear texture), and mean lesser diameter.

FIG. 7 is a Kaplan-Meier curve for 157 stage Ta NMIBC patients grouped by the percentage of interquartile range (IQR) outliers for nuclear perimeter stratified by the median (p=0.042).

FIG. 8 is a receiver operator curve of a K-Nearest Neighbour model predicting HG from LG tumours using the percentage of IQR outliers for nuclear size features (AUC=0.865).

FIGS. 9A-9H are images showing the top three quantitative nuclear imaging features indicating risk of earlier recurrence, wherein FIGS. 9A-9C show low to high mean lesser diameter, a measure of nuclear size and shape; FIG. 9D shows high mitotic index, a marker of proliferation; FIGS. 9E-9H show high to low mean variance HEM.

FIGS. 10A and 10B are plots of (A) Brier score and (B) continuous rank probability score (CRPS) curves across time comparing a basic Kaplan-Meier model to the RSF model using histological features.

### DETAILED DESCRIPTION OF EMBODIMENTS

Described herein are histology-based grading and prognostic methods that use measurements of cancer cell features mapped to time-dependent outcomes rather than recurrence status at a standard time point. Given the quantitative nature of this strategy, embodiments may be applied consistently across cohorts, they can more objectively and accurately inform care compared to standard grading strategies, and they can be tied to biological mechanistic relevance. Embodiments may be based on statistical and machine learning models that use quantitative histologic imaging features to predict and score risk of recurrence, expressed as estimated time to cancer recurrence with a selected confidence interval, e.g., a 95% confidence interval. Compared to current clinical risk scoring strategies, embodiments provide superior prognostic performance. Providing an estimated time to recurrence according to embodiments described herein may provide more precise guidance for treating and monitoring patients, for example, for determining an appropriate interval for cystoscopic surveillance, tailored to precise measurements of each patient's cancer cell nuclei.

Embodiments are described herein primarily with respect to non-muscle invasive bladder cancer (NMIBC). However, it will be appreciated that the invention is not limited thereto, as the methods may be extended and adapted to quantify and leverage the prognostic value of histologic imaging data to predict time to recurrence and determine other relationships in other cancers (e.g., noninvasive papillary urothelial carcinoma, thyroid, adrenal (cortical and medullary), neuroendocrine, endometrial, astrocytic, renal prostate, breast, and colorectal, soft tissue sarcoma, and lung,), which may be used to support pathologist decision making.

According to embodiments, analyses examine the use of quantified, grade-based imaging features to construct statistical and machine learning survival models of recurrence in patients. Findings presented herein highlight the prognostic value of key quantitative nuclear features (QNFs), and support the use of a continuous, numerical histology-based prognostic score for each patient. To properly handle time-dependent prognostic data, embodiments may employ survival models such as, for example, Cox Proportional Hazards (CPH) regression and Random Survival Forests (RSF), and corresponding performance evaluation of concordance (C)-index.

In certain embodiments, CPH and RSF models for recurrence-free survival were constructed using QNFs extracted from bladder cancer tissue digital photomicrographs using machine learning (ML) based image analysis software. Models built using grade-based QNFs were compared to models of subjective, pathologist-determined grade, as well as American Urological Association (AUA) risk score, the current clinical standard risk stratification tool, and provided superior results. The results demonstrate the prognostic value of key QNFs, and demonstrate the use of a continuous, numerical histology-based prognostic score for each patient.

Given the improved performance when using QNF-based features over grade, a strategy as described herein for quantitative histology-based risk scoring also provides pathologists with a decision support tool that can eliminate several detrimental sources of data loss. Rather than subjective visual assessment in standard grading, obtaining measurements for features of interest provides a more complete and reliable assessment of the tissue. Utilizing a continuous risk score eliminates the data loss associated with setting an arbitrary threshold between low- and high-grade tumours. Nuclear feature measurements may be weighted based on their importance for outcomes of interest, so that the most important features may be prioritized and to continuously tune the grading strategy towards optimal prognostic value.

Whereas survival analysis and modelling has been considered in studies of NMIBC prognostics [e.g., 14-20], some have focused on fewer features, whereas others have used a larger number of features but they were all derivatives of fewer categories. For example, without analyzing mitotic figures, which were excluded from their study, Tokuyama found that features important in classification models for NMIBC recurrence were based on only shape and texture, however, they identified 79 categories within those features for use in analyses. In contrast, the methods described herein use only a small number of features but with a broad spectrum in that they are related to diverse characteristics of, e.g., size, shape, mitotic index, and texture. In addition, the embodiments described herein demonstrate the importance of including time- and censoring-related aspects to recurrence prediction in NMIBC rather than simply classifying recurrence status as a binary yes or no. Also, in the literature, AUA risk score is shown to carry prognostic value in terms of time-to-recurrence in NMIBC including both stage Ta and T1 [22]. However, as shown in FIG. 5, the AUA risk score does a poor job of distinguishing high-risk and intermediate-risk cases in the stage Ta cohort, where stage is not a differentiating factor.

Embodiments will be further described by way of the following non-limiting example.

### Example 1

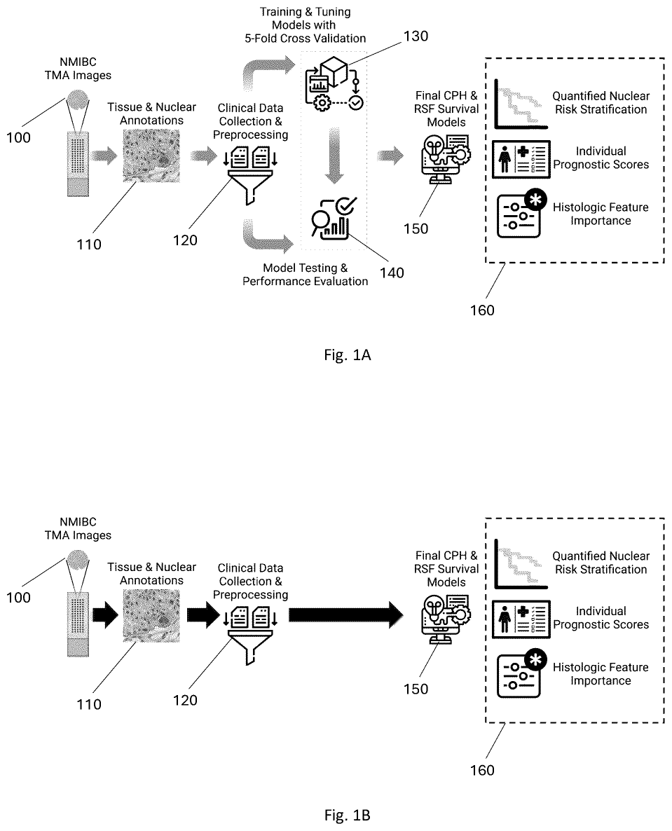

FIG. 1A is a schematic representation of a workflow that includes training of the image recognition algorithm. The workflow may be applied to and/or adapted for types of cancer other than NMIBC. Briefly, referring to FIG. 1A, tissue microarrays (TMAs) 100 with one or two 1 mm diameter images were prepared from tumour samples. They were analyzed with imaging software that was trained to measure QNFs based on tissue and nuclear annotations 110. Clinical data was collected and preprocessed for each tissue sample 120. Prognostic survival models combining the QNFs with clinical data were trained 130 and evaluated 140. Outcomes of the final survival models 150 and the model development process include quantified nuclear risk stratification, individual prognostic score, and histologic feature importance 160.

FIG. 1B is a schematic representation of a workflow wherein the image recognition software is trained to measure QNFs, as may be used in a clinical setting to evaluate a digital histology image of tumour cells of a patient to determine a prognostic score for the patient. The same reference numerals are used for the same or similar features as in FIG. 1A. In a clinical setting, one or more of the features shown at 160 in FIG. 1B may be implemented.

### Example 2

This example describes using grading criteria as quantitative nuclear features (QNFs) and employing Al-driven image analysis to extract QNFs, using the QNFs as building blocks for prognostic classifiers, and using QNFs to externally validate grading models and create recurrence-free survival (RFS) models in NMIBC.

### Cohort Data Collection

This study was approved by the Queen's University Research Ethics Board. The Kingston Health Sciences Centre pathology database was queried for patients diagnosed with NMIBC between 2008-2016 [6], with longitudinal clinical timeline data retrospectively collected until 2022. One millimeter diameter digital photomicrographic images were generated to provide imaging data representative of each tumour sample while maintaining a low data volume compared to whole slide images (WSIs). This approach improved the efficiency and efficacy of the pathology workflow. Consensus re-grading for every image was performed by presenting each image to three separate reviewers to obtain an image-specific diagnosis based on the World Health Organization (WHO) 2004/2016 NMIBC grading criteria. This limited the impact of grade heterogeneity within each tumour, providing the opportunity to analyse a small area of tissue representative of what is expected to be driving the behaviour of the tumour as a whole.

### Image Analysis and Feature Extraction

The TMA images were segmented, and the tissue was annotated for tumour, non-tumour, and glass using machine learning software (Visiopharm®, Hoersholm, Denmark).

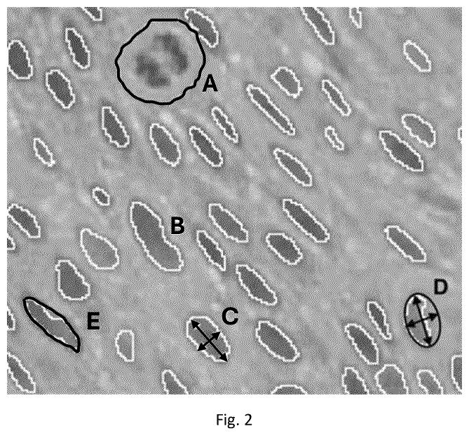

Manual correction of inaccurate segmentation was performed, excluding tumour artifact and separating lymphoid structures [23]. Algorithms were developed and trained within the software to obtain morphometric measurements and detect mitotic figures [23]. Referring to FIG. 2, morphometric features included: A, mitotic figure; B, using the pixelated contour line (white), area, perimeter, and form factor (4*π*area/perimeter²); C, lesser and largest-diameters of the nucleus to the perimeter; D, major and minor axes (length and width measurements to the nearest ellipse), ellipticalness, and eccentricity; E, solidity and convexity (area (or perimeter)/bounding polygon size, as determined by polygon simplification). Nuclear segmentation using the Visiopharm pre-trained automatic segmentation classifier enabled the extraction of nuclear morphometry data. A mitotic figure detection routine was developed using the Visiopharm U-net deep-learning algorithm with initial manual identification of approximately 200 mitotic figures by the inventors, with labelling correction as needed, and repeating this process multiple times. A list of the quantitative nuclear feature outputs is presented in Table 1.

TABLE 1
Quantitative Nuclear Features extracted
from digital photomicrographic images.
Quantitative Nuclear Features

| | |
|---|---|
| Mean Area | SD Convexity |
| Mean Convexity | SD Eccentricity |
| Mean Eccentricity | SD Ellipticalness |
| Mean Ellipticalness | SD Form Factor |
| Mean Form Factor | SD Larger Diameter |
| Mean Larger | SD Lesser Diameter |
| Diameter | SD Perimeter |
| Mean Lesser Diameter | SD Solidity |
| Mean Perimeter | SD Variance HEM |
| Mean Solidity | Fraction Mitotic Figures |
| Mean Variance HEM | Mitotic Index |

### Data Preprocessing

Clinical data preprocessing was performed using R version 4.2.2 (r-project.org). A summary of the preprocessing steps is presented in FIG. 3, which is a flowing chart showing how data were filtered, resulting in the final data that was used to train the models and develop the workflow. First, the digital photomicroph imaging data was reduced to one representative image per patient. For samples with two representative images with disagreeing grades, the lower graded image was excluded. For samples where both images were assigned the same grade and the feature measurements were highly correlated, the mean of each measured feature was calculated, weighted by the number of nuclei in each image to obtain a representative set of measurements for the sample. The data was randomly partitioned into a training set and a testing set by an 80/20 split using the caret package in R [7], balanced for recurrence status, progression status, sample grade, and AUA risk score. Min-max feature scaling was applied to obtain a standard nuclear measurement range between 0-1 based on the distribution of the training set.

In summary, the study included patients who received an initial diagnosis of stage Ta NMIBC, where a bacillus Calmette-Guerin (BCG) treatment-naïve transurethral resection of bladder tumour (TURBT) sample was available and met histological imaging quality requirements. Clinical and demographic information for the 163 patients that met inclusion criteria is presented in Table 2. Recurrence free survival (RFS) was defined as time from NMIBC TURBT resection to next TURBT with pathological confirmation of bladder cancer, including low-grade disease. Cystoscopy and operative notes were reviewed to exclude re-resections as recurrences, defined by subsequent TURBT within 42 days of initial TURBT. Censoring was applied to patients alive and without recurrence at last-follow-up, or who died without experiencing a recurrence with the censoring event being either last follow-up or non-bladder cancer related death.

TABLE 2
Cohort characteristics relevant to time-to-recurrence survival analysis.

| | | |
|---|---|---|
| Low Grade | High Grade | Total |
| (N = 113) | (N = 50) | (N = 163) |

Sex

| | | | | | | |
|---|---|---|---|---|---|---|
| Female | 33 | (29.2%) | 7 | (14.0%) | 40 | (24.5%) |
| Male | 80 | (70.8%) | 43 | (86.0%) | 123 | (75.5%) |

Age (years)

| | | | | | | |
|---|---|---|---|---|---|---|
| Mean (SD) | 69.3 | (11.2) | 73.6 | (9.96) | 70.6 | (11.0) |
| Median [Min, Max] | 70.0 | [34.0, 89.0] | 74.5 | [49.0, 94.0] | 71.0 | [34.0, 94.0] |

Recurrence

| | | | | | | |
|---|---|---|---|---|---|---|
| No Recurrence | 58 | (51.3%) | 21 | (42.0%) | 79 | (48.5%) |
| Recurrence | 55 | (48.7%) | 29 | (58.0%) | 84 | (51.5%) |

Years to First Recurrence

| | | | | | | |
|---|---|---|---|---|---|---|
| Median [Min, Max] | 1.31 | [0.153, 11.8] | 0.605 | [0.145, 5.64] | 0.930 | [0.145, 11.8] |

Total Recurrences

| | | | | | | |
|---|---|---|---|---|---|---|
| Mean (SD) | 1.41 | (2.03) | 1.48 | (1.76) | 1.43 | (1.95) |
| Median [Min, Max] | 1.00 | [0, 12.0] | 1.00 | [0, 8.00] | 1.00 | [0, 12.0] |

Progression

| | | | | | | |
|---|---|---|---|---|---|---|
| No Progression | 95 | (84.1%) | 37 | (74.0%) | 132 | (81.0%) |
| Progression | 18 | (15.9%) | 13 | (26.0%) | 31 | (19.0%) |

Years to Last Follow-Up (years)

| | | | | | | |
|---|---|---|---|---|---|---|
| Median [Min, Max] | 6.15 | [0, 25.5] | 5.53 | [0.00848, 13.1] | 5.88 | [0, 25.5] |

### Cox Proportional Hazards (CPH) Model

The proportional hazards, independent censoring, and non-collinearity assumptions were assessed in R using the survival package [8] on the training dataset in advance of feature selection and model construction. Features that did not pass the assumptions were excluded from the CPH model. To construct the final multivariable CPH model, added value of each histological feature was assessed by comparing the baseline model of the top performing feature in univariable analysis to models with each combination of added features using the Likelihood Ratio Test (LRT). If the model with the additional feature showed a significant improvement in predictive power from the baseline model, the feature was added to the final model.

### Random Survival Forest (RSF) Model

From the 22 grade-based histology features (Table 1), redundant features were eliminated from the set if they surpassed the variance inflation factor threshold to limit multicollinearity in the model [9]. To avoid overfitting of the model, the number of features used in the model was reduced to one tenth of the number of events, in this case recurrences, in the training set. The random forest minimum depth feature selector was used in the model to set a threshold based on variable predictiveness to separate useful variables from noisy variables [10, 11].

The RSF model was built using the R package randomForestSRC [12]. This method grows trees using bootstrapped data with random selection of features at each tree node, split such that daughter nodes are optimally separated by survival behaviour using a log-rank split. Hyperparameters were tuned using the tuning functions in the randomForestSRC package during the training phase. The resulting survival forest ensemble provides the average of terminal node statistics of survival function and cumulative hazard function based on Nelson-Aalen and Kaplan-Meier estimates.

### Validation and Statistical Analysis

The CPH and RSF models were constructed using quantitative nuclear features. For both models, 5-fold cross-validation was used during training. The training set was split into five folds, where separate models were constructed on each possible combination of four training folds and evaluated on the fifth fold. Performance metrics were recorded for each of the five training models and averaged to get overall performance metrics for the training set. The top performing models for each strategy, defined by the best Harrell's C-index, from the cross-validation training step was then used for evaluation with the test set. For the RSF model, the Brier score and continuous rank probability score (CRPS) were also plotted to compare model predictions over time to ground-truth [13].

For both the CPH and the RSF models, bootstrapping was employed to obtain 95% confidence intervals for the C-index in the testing set. For 1,000 iterations, a random sample the size of the test set was selected with replacement to run through the model. Harrell's C-indices from each of the 1,000 iterations were ordered and the 25^{th} and 975^{th} C-indices were used as confidence intervals for the testing set. Harrell's C-index was also calculated for CPH models built using current clinical standard indicators of prognostic risk including grade and American Urological Association (AUA) risk score to compare histological feature models to the current clinical standard.

### Interquartile Range (IQR) Detector

An interquartile range (IQR)-based outlier detector was developed using a reference cohort of five low-grade samples (38,702 nuclei). A threshold was set to mark abnormally large or abnormally small nuclear features as outliers. An outlier score was developed indicating the percent of abnormal nuclear size features (e.g., percent outliers of one or more nuclear feature selected from area, convexity, eccentricity, ellipticalness, form factor, perimeter, lesser diameter, larger diameter, solidity and variance HEM) present in a sample. Feature selection was performed to identify set of features that best discriminate between high-grade and low-grade tumours and between recurred and non-recurred samples. Using selected features, machine learning models were trained to discriminate between high-grade and low-grade tumours and to predict recurrence.

### Results

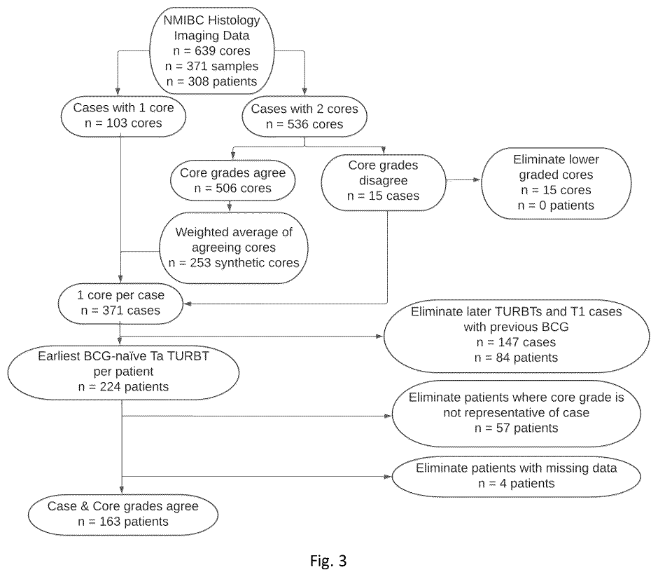

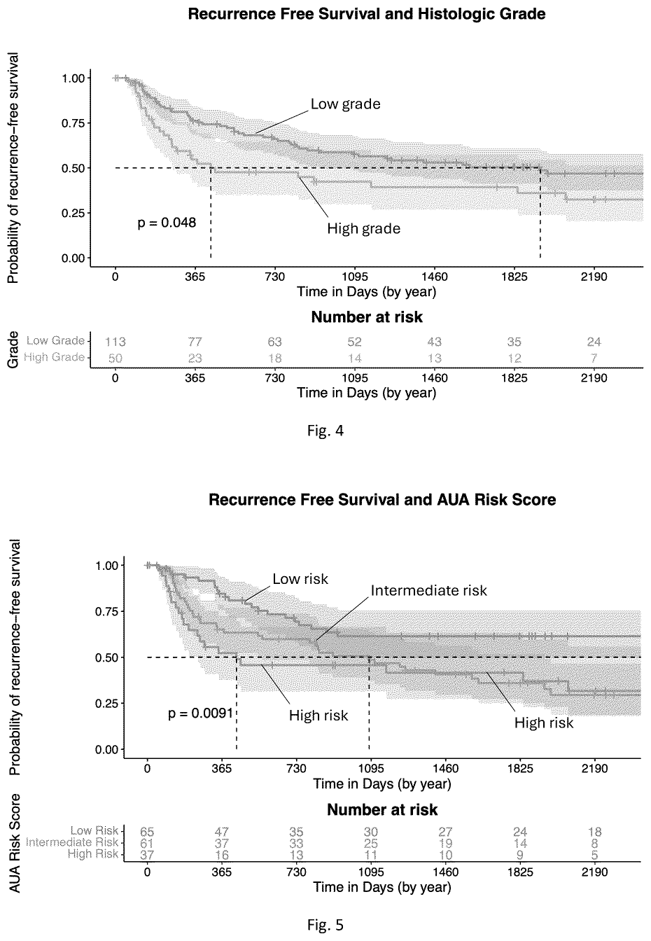

As noted above, 163 stage Ta NMIBC patients met the final inclusion criteria for the survival study. A total of 84 patients experienced at least one recurrence, with median time to first recurrence of 0.605 years for high-grade cases (n=29 of 50 high-grade cases) and 1.31 years for low-grade cases who recurred (n=55 of 113 low-grade cases). FIG. 4 is a Kaplan-Meier curve for stage Ta NMIBC patients stratified by grade. FIG. 5 is a Kaplan-Meier curve with the cohort stratified by AUA risk score. Characteristics of the cohort regarding outcomes relevant to time-to-event survival analyses are presented in Table 2.

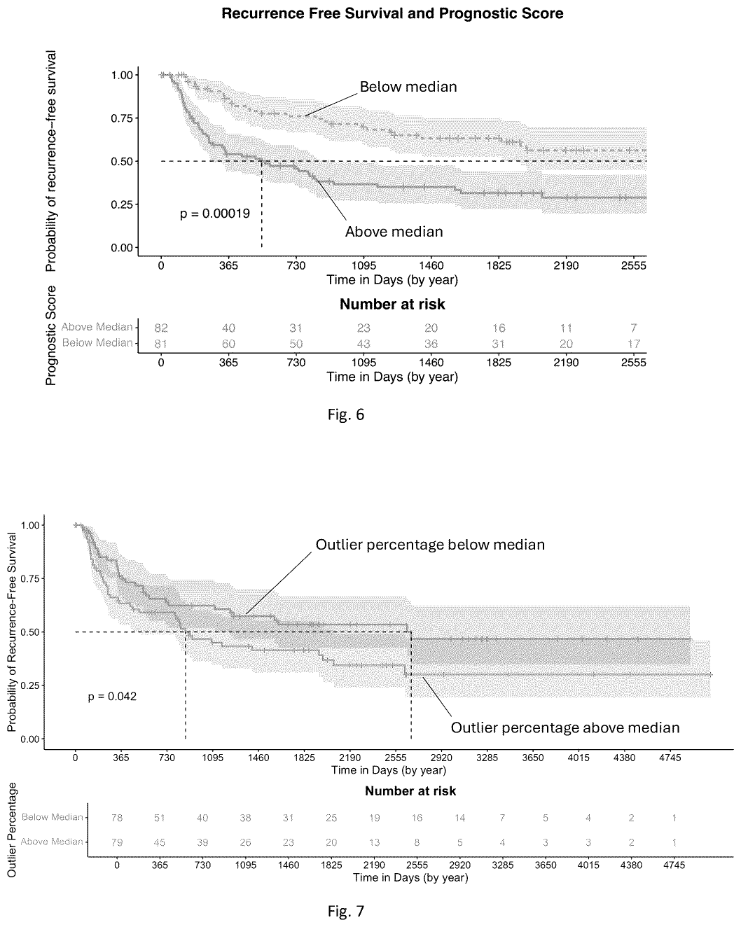

Univariable CPH analysis of histologic and clinical features are presented in Table 3. Multivariable model performance by Harrell's C-index for clinical standard models and for quantitative nuclear feature (QNF) models are presented in Table 4. CPH models estimate the optimal coefficient for each feature when calculating an individual patient's prognostic score. These prognostic scores were calculated for each patient based on the coefficients optimized using the CPH QNF model. The median of the prognostic score distribution was used as a stratification threshold for the Kaplan-Meier curves in FIG. 6, where separation was assessed using the log-rank test.

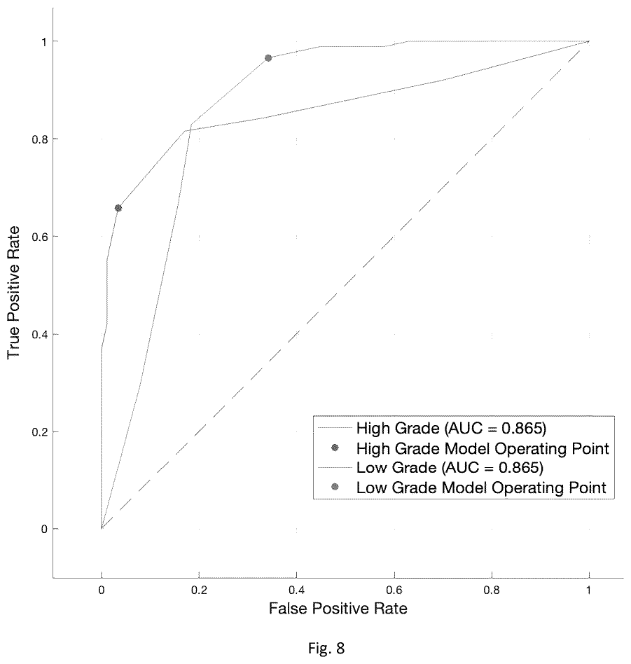

Results for the IQR-based outlier detecter are shown in FIGS. 7 and 8. FIG. 7 is a Kaplan-Meier curve for 157 stage Ta NMIBC patients grouped by the percentage of IQR outliers for nuclear perimeter stratified by the median (p=0.042). FIG. 8 is a receiver operator curve of a K-Nearest Neighbour model predicting HG from LG tumours using the percentage of IQR outliers for nuclear size features with a validation accuracy of 87.3% (AUC=0.865).

TABLE 3
Univariable CPH analysis of demographic features, histologic features
that pass CPH assumptions, and AUA risk scoring features.

| | Beta (β) | HR | | Wald | |
|---|---|---|---|---|---|
| | Coefficient | (95% CI) | | Test | (p-value) |
| Sex | 0.21 | 1.2 | (0.75-2) | 0.70 | 0.4 |
| Age | 0.018 | 1 | (1-1) | 2.70 | 0.1 |
| Grade | 0.45 | 1.6 | (1-2.5) | 3.90 | 0.049 |
| Mean Eccentricity | −0.35 | 0.7 | (0.22-2.3) | 0.35 | 0.55 |
| Mean Lesser Diameter | 0.86 | 2.4 | (0.65-8.5) | 1.70 | 0.19 |
| Mean Variance HEM | −1.3 | 0.26 | (0.082-0.85) | 5.00 | 0.026 |
| SD Eccentricity | 0.6 | 1.8 | (0.57-5.8) | 1.00 | 0.31 |
| SD Form Factor | 0.52 | 1.7 | (0.66-4.3) | 1.20 | 0.28 |
| SD Lesser Diameter | 0.75 | 2.1 | (0.77-5.8) | 2.10 | 0.15 |
| Mitotic Index | 1.6 | 5.1 | (2-13) | 11.00 | 0.00071 |
| AUA Risk Score | 0.81 | 2.2 | (1.3-3.9) | 8.20 | 0.0041 |
| Tumour Multifocality | 0.54 | 1.7 | (1.1-2.7) | 6.00 | 0.014 |
| Tumour Size | 0.73 | 2.1 | (1.3-3.4) | 8.50 | 0.0036 |

TABLE 4
Model Performance for Recurrence Free Survival,
histologic features include Mitotic Index,
Mean Variance HEM, and Mean Lesser Diameter.

| | |
|---|---|
| Train C-Index | Test C-Index (95% CI) |

Cox Proportional Hazards Model

| | | |
|---|---|---|
| Grade | 0.57 | 0.55 (0.40-0.69) |
| AUA Risk Score | 0.61 | 0.58 (0.43-0.71) |
| Histologic Features | 0.65 | 0.73 (0.56-0.88) |

Random Survival Forest Model

| | | |
|---|---|---|
| Histologic Features | 0.62 | 0.70 (0.66-0.70) |

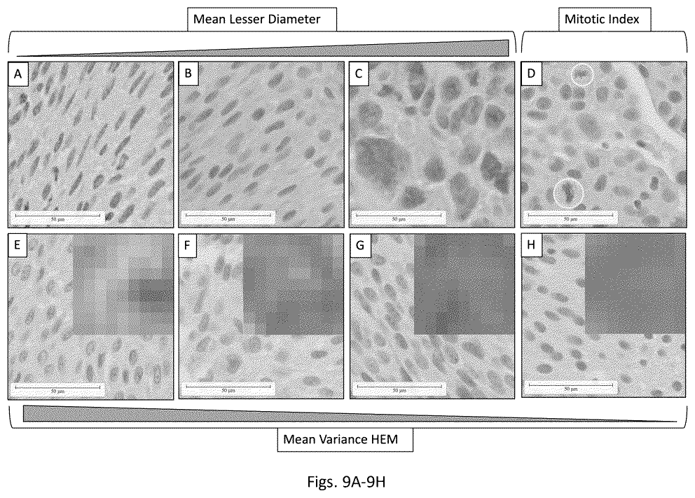

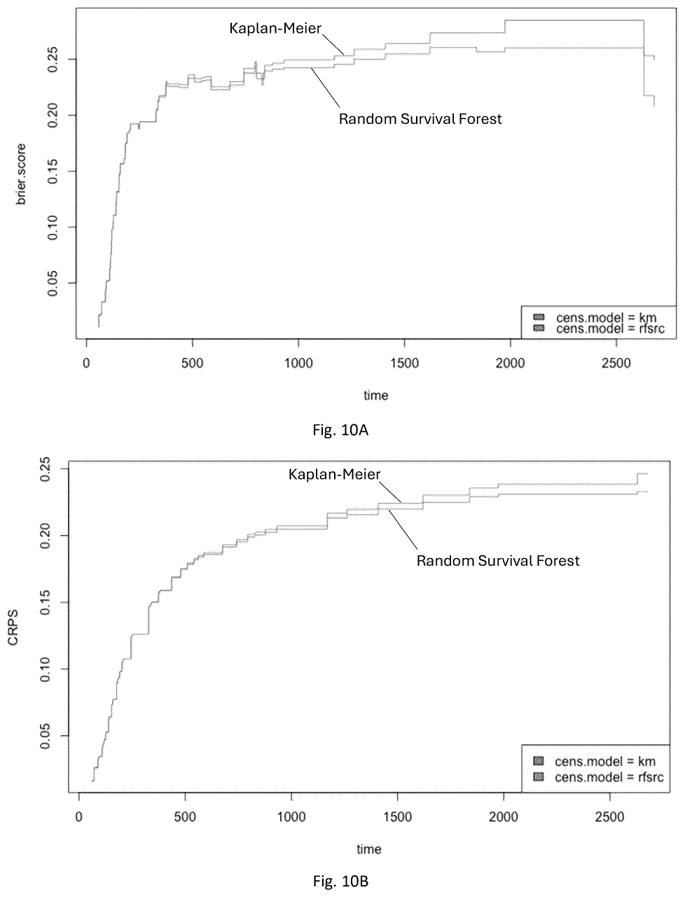

Feature selection based on minimum depth and variable importance selected the same three features as most correlated with recurrence-free survival: low to high mean lesser diameter, a measure of nuclear size and shape (e.g., FIGS. 9A-9C); high mitotic index, a marker of cell proliferation (e.g., FIG. 9D); high to low mean variance HEM, a measure of staining intensity for nuclear texture with more homogenously textured nuclei marking increased chromatin (e.g., FIGS. 9E-9H). RSF model performance on out-of-bag samples for both the training 5-fold cross-validation and test set are shown in Table 4 with all other model performance metrics. Brier scores of the RSF model compared to a general Kaplan-Meier analysis on the training set are plotted in FIG. 10A, where values closer to 0 indicate better model performance. The continuous rank probability score (CRPS), an extension of the Brier score as a time-specific metric similar to mean absolute error for use in probabilistic forecasting, is plotted in FIG. 10B comparing a typical Kaplan-Meier model with the RSF model.

In validation images, CPH recurrence-free survival models constructed using the selected grade-based QNFs (mitotic index, mean variance HEM, and mean lesser diameter) outperformed models based on grade and standard risk-scoring procedures, achieving a C-index of 0.73 compared to 0.55 for grade and 0.58 for AUA risk score. The same QNFs were selected for and used in the RSF model, which achieved a C-index of 0.70 with a much narrower 95% confidence interval range (FIG. 6).

When the cohort was stratified by the median QNF-based prognostic score, Kaplan-Meier curves showed better separation than models using pathologist-assigned grade. The improved model performance of QNF-based prognostic scoring from standard risk tools was also indicated by higher C-indices. These results demonstrate the clinical and prognostic value of methods described herein based on quantifying and standardizing grading classification strategies that establish prognostic feature importance weights and utilize continuous, numeric risk scores, thereby providing a significant improvements over prior subjective approaches. The results indicate that stage Ta NMIBC histology images carry rich prognostic data and embodiments demonstrate how the data can be extracted and used to improve clinical decision making and care. Using embodiments described herein, a prognostic score for a patient may be used to determine whether the patient should receive further treatment, and if so, the type of treatment, etc. For example, a prognostic score indicating a high risk for recurrence for a patient may suggest that a certain type of treatment would be appropriate to reduce the risk of recurrence, where such treatment would otherwise not be considered for the patient in the absence of an analysis as described herein.

All cited publications are incorporated herein by reference in their entirety.

### EQUIVALENTS

It will be appreciated that modifications may be made to the embodiments described herein without departing from the scope of the invention. Accordingly, the invention should not be limited by the specific embodiments set forth, but should be given the broadest interpretation consistent with the teachings of the description as a whole.

### REFERENCES

1. Soukup V, Capoun O, Cohen D, et al: Prognostic Performance and Reproducibility of the 1973 and 2004/2016 World Health Organization Grading Classification Systems in Non-muscle-invasive Bladder Cancer: A European Association of Urology Non-muscle Invasive Bladder Cancer Guidelines Panel Systematic Review. Eur Urol 72:801-813, 2017
2. Chao C M, Yu Y W, Cheng B W, et al: Construction the model on the breast cancer survival analysis use support vector machine, logistic regression and decision tree. J Med Syst 38:106, 2014
3. Deist T M, Dankers F, Valdes G, et al: Machine learning algorithms for outcome prediction in (chemo) radiotherapy: An empirical comparison of classifiers. Med Phys 45:3449-3459, 2018
4. Tokuyama N, Saito A, Muraoka R, et al: Prediction of non-muscle invasive bladder cancer recurrence using machine learning of quantitative nuclear features. Mod Pathol 35:533-538, 2022
5. George B, Seals S, Aban I: Survival analysis and regression models. J Nucl Cardiol 21:686-94, 2014
6. Jackson C L: Prognostic Features of Non-muscle-invasive Bladder Cancer: Grade, Molecular Subtype and Tumour Immune Microenvironment, Queen's University (Canada), 2021
7. Kuhn M: The caret package. R Foundation for Statistical Computing, Vienna, Austria. URL https://cran.r-project.org/package=caret, 2012
8. Therneau T: A Package for Survival Analysis in R, R Package Version 3.5-0, R Foundation Vienna, Austria, 2023
9. Belsley D A: A guide to using the collinearity diagnostics. Computer Science in Economics and Management 4:33-50, 1991
10. Spooner A, Chen E, Sowmya A, et al: A comparison of machine learning methods for survival analysis of high-dimensional clinical data for dementia prediction. Sci Rep 10:20410, 2020
11. Ishwaran H, Chen X, Minn A J, et al: randomForestSRC: Minimal Depth Vignette. 2021
12. Ishwaran H, Lauer M S, Blackstone E H, et al: Randomforestsrc: Random survival forests vignette, 2021
13. Gerds T A, Schumacher M: Consistent estimation of the expected Brier score in general survival models with right-censored event times. Biom J 48:1029-40, 2006
14. Lujan S, Santamaria C, Pontones J L, et al: Risk estimation of multiple recurrence and progression of non muscle invasive bladder carcinoma using new mathematical models. Actas Urol Esp 38:647-54, 2014
15. Soria F, D'Andrea D, Abufaraj M, et al: Stratification of Intermediate-risk Non-muscle-invasive Bladder Cancer Patients: Implications for Adjuvant Therapies. Eur Urol Focus 7:566-573, 2021
16. Fukuokaya W, Kimura T, Miki J, et al: Red cell distribution width predicts time to recurrence in patients with primary non-muscle-invasive bladder cancer and improves the accuracy of the EORTC scoring system. Urol Oncol 38:638 e15-638 e23, 2020
17. Laukhtina E, Mostafaei H, D'Andrea D, et al: Association of De Ritis ratio with oncological outcomes in patients with non-muscle invasive bladder cancer (NMIBC). World J Urol 39:1961-1968, 2021
18. Egbers L, Grotenhuis A J, Aben K K, et al: The prognostic value of family history among patients with urinary bladder cancer. Int J Cancer 136:1117-24, 2015
19. van Rhijn B W G, Hentschel A E, Brundl J, et al: Prognostic Value of the WHO1973 and WHO2004/2016 Classification Systems for Grade in Primary Ta/T1 Non-muscle-invasive Bladder Cancer: A Multicenter European Association of Urology Non-muscle-invasive Bladder Cancer Guidelines Panel Study. Eur Urol Oncol 4:182-191, 2021
20. Daza J, Grauer R, Chen S, et al: Development of a predictive model for recurrence-free survival in pTa low-grade bladder cancer. Urol Oncol, 2023
21. Tokuyama N, Saito A, Muraoka R, et al: Prediction of non-muscle invasive bladder cancer recurrence using machine learning of quantitative nuclear features. Mod Pathol, 2021
22. Ritch C R, Velasquez M C, Kwon D, et al: Use and Validation of the AUA/SUO Risk Grouping for Nonmuscle Invasive Bladder Cancer in a Contemporary Cohort. J Urol 203:505-511, 2020
23. Slotman A, Xu M, Lindale K, et al: Quantitative nuclear grading: an objective, artificial intelligence-facilitated foundation for grading noninvasive papillary urothelial carcinoma. Lab Invest: 100155, 2023

1. A computer-implemented method for classifying cancer, comprising:
utilizing image analysis software to analyze a digital histology image of tumour cells of a patient, the image analysis software being trained to segment tissue regions, identify nuclei, and measure nuclear features of a plurality of nuclei;
obtaining summary statistics for nuclear feature values;
applying one or more prognostic classifier to the nuclear feature values; and
producing a prognostic score for the patient from the one or more prognostic classifier.

2. The method of claim 1, wherein the nuclear features are selected from size, shape, texture, and mitotic index.

3. The method of claim 1, wherein the nuclear features for nuclei in an image are reduced into summary statistics including mean and standard deviation.

4. The method of claim 1, wherein tumour and non-tumour regions are segmented, wherein only tumour nuclei are included in prognostic classifiers.

5. The method of claim 1, comprising using multiple histology images for a single patient and the summary statistics of the nuclear features are a weighted average of the multiple histology images.

6. The method of claim 1, wherein the prognostic score is at least one of recurrence-free survival of the patient and discrimination between high-grade and low-grade tumours.

7. The method of claim 1, wherein the cancer is non-muscle invasive bladder cancer.

8. The method of claim 1, wherein the one or more prognostic classifier comprises a Cox Proportional Hazards (CPH) model.

9. The method of claim 1, wherein the one or more prognostic classifier comprises a Random Survival Forest (RSF) model.

10. The method of claim 1, wherein the one or more prognostic classifier comprises an interquartile range (IQR)-based outlier detector.

11. The method of claim 10, wherein the IQR-based outlier detector determines an outlier score indicating a percent of abnormal size for at least one nuclear size feature.

12. The method of claim 1, wherein the prognostic score for the patient is used to determine the appropriateness and type of treatment for the patient.

13. Non-transitory computer readable media for use with a processor, the computer readable media having stored thereon instructions that when executed by the processor, cause the processor to execute processing steps comprising:
executing an algorithm trained to analyze a digital histology image of tumour cells of a patient, including segmenting tissue regions, identifying nuclei, and measuring nuclear features of a plurality of nuclei;
determining summary statistics for the nuclear feature values;
applying one or more prognostic classifier to the nuclear feature values; and
producing a prognostic score for the patient from the one or more prognostic classifier.

14. The non-transitory computer readable media of claim 13, wherein the nuclear features are selected from size, shape, texture, and mitotic index.

15. The non-transitory computer readable media of claim 13, wherein the nuclear features for nuclei in an image are reduced into summary statistics including mean and standard deviation.

16. The non-transitory computer readable media of claim 13, wherein tumour and non-tumour regions are segmented, wherein only tumour nuclei are included in prognostic classifiers.

17. The non-transitory computer readable media of claim 13, wherein the prognostic score is at least one of recurrence-free survival of the patient and discrimination between high-grade and low-grade tumours.

18. The non-transitory computer readable media of claim 13, wherein the one or more prognostic classifier comprises a Cox Proportional Hazards (CPH) model.

19. The method of claim 1, wherein the one or more prognostic classifier comprises a Random Survival Forest (RSF) model.

20. The non-transitory computer readable media of claim 13, wherein the one or more prognostic classifier comprises an interquartile range (IQR)-based outlier detector.

21. The non-transitory computer readable media of claim 20, wherein the IQR-based outlier detector determines an outlier score indicating a percent of abnormal size for at least one nuclear size feature.

22. The non-transitory computer readable media of claim 13, wherein the prognostic score for the patient is used to determine the appropriateness and type of treatment for the patient.
